(19) United States
(12) Patent Application Publication
(10) Pub. No.: US 2024/0180730 A1
(43) Pub. Date: Jun. 6, 2024

(54) INTESTINAL SLEEVE

(71) Applicant: Allevetix Medical Ltd., Ceasarea (IL)

(72) Inventor: Ziv KALFON, Ein Hod (IL)

(21) Appl. No.: 18/553,609

(22) PCT Filed: Apr. 8, 2021
(86) PCT No.: PCT/IL2021/050411

§ 371 Date: Oct. 2, 2023

(51) Int. Cl. A61F 5/00

(52) U.S. Cl. CPC A61F 5/0076; A61F 5/0013; A61F 2005/002

(57) ABSTRACT

A gastrointestinal device that is inserted into the intestine and includes a tension sensor and an RFID transmitter. The tension sensor senses the strain applied by the intestinal sleeve and pylorus anchor to the stomach's anchor. The RFID transmits the tension to an outside receiver that allows the caregiver or user to monitor the intestinal sleeve and pylorus anchor location.

### TECHNOLOGY FIELD

The present sleeve is related to a device inserted into a body cavity using intraoral procedures and particularly to a device that maintains the intestinal sleeve position within a body cavity.

### BACKGROUND

The use of gastrointestinal implant devices and, in particular, bariatric devices is believed to be an effective treatment for obesity and type 2 diabetes and several other obesity-related conditions. The bariatric devices are adapted to be inserted intraorally into the stomach. The inserted devices extend from the stomach into the intestines. All food exiting the stomach is funneled through the implant device.

A gastrointestinal implant device includes an unsupported flexible, collapsible sleeve and an anchor. The anchor is usually coupled to a proximal to the stomach portion of the sleeve. The flexible sleeve is open at both ends and adapted to extend into the small intestine and, particularly, to the duodenum to limit the absorption of nutrients in the small intestine and duodenum. The anchor is adapted to secure and retain the sleeve within the duodenum. In some examples, there could be an additional anchor residing in the stomach. The sleeve could be of constant or variable length.

The anchors stabilize the inserted medical device and restrain the movement of the inserted medical device. U.S. Pat. No. 9,636,245 to Chamorro describes a gastrointestinal device including a proximal element configured to reside in the stomach and a distal element configured to reside in an intestine. The proximal element is configured to resist migration over time. US Pat. Application No. 2008/0058840 to Albrecht describes an alternative proximal element for placement within a hollow body organ. The proximal element includes a member having a first shape for delivery to the hollow body and a second shape for implantation. The member has sufficient rigidity in its second shape to exert an outward force against the hollow body's interior to bring together two substantially opposing surfaces of the hollow body. US Pat. Application No. 2012/0095385 to Domingues describes a gastric balloon that is introduced in the stomach. The balloon occupies space in the stomach, leaving less room for food and creating a patient's satiety. U.S. Pat. No. 6,994,715 to Gannoe discloses expandable and space-occupying devices inserted into the stomach of a patient and may be maintained within the stomach by anchoring or otherwise fixing the device to the stomach wall. Patent Cooperation Treaty Publication No. 2007/076021 to Haller describes a gastrointestinal device, which is a bag stuffed with digestive resistant or indigestible material. The bag is located in the gastric lumen in a compacted configuration. The bag is then manipulated and allowed to assume a second expanded configuration sufficiently large to maintain the bag within the stomach and not to be passed through the pylorus and into the intestines. The implants described in the listed above patents suffer from certain drawbacks when inserted within a human body's lumen. They do not provide the long-term required implantation stability.

Small intestine length being in contact with the digested food or chime determines the caloric absorptive capacity. The gastrointestinal implant device lining the intestine limits the small bowel length being in contact with the digested food. It receives from the stomach the digested food and delivers it 30-100 cm down the intestines and delays the breakdown and subsequent digestion of food.

Different gastrointestinal devices, including intestinal sleeves, are also described in several United States patents, including U.S. Pat. No. 8,855,770 to Gross et al.; U.S. Pat. No. 8,888,732 to Raven et al.; U.S. Pat. No. 8,956,380 to Dominguez et al.; U.S. Pat. No. 9,011,365 to Connor; U.S. Pat. No. 9,173,734 to Vargas; U.S. Pat. No. 9,289,580 to Coleman et al.; U.S. Pat. No. 9,463,107 to Babkes et al.; U.S. Pat. No. 9,504,591 to Burnett et al.; U.S. Pat. No. 9,681,974 to Dominguez et al.; U.S. Pat. No. 9,717,584 to Culley et al.; and United States Patent Application Publications 20030114803 to Lerner; 20120232459 to Dann et al.; 20140316265 to Levin et al. and 20170181877 to Binmoeller.

### SUMMARY

This current disclosure describes a gastrointestinal implantable device including a stomach anchor, a pylorus anchor, an intestinal sleeve, and a device monitoring the pylorus anchor and the intestinal sleeve location. The implantable device is inserted into a body cavity using intraoral procedures.

The gastrointestinal device includes at least one tension sensor and at least one radio wave transmitter. The tension sensor senses tension caused by migration of the gastrointestinal sleeve and a pylorus anchor and communicates the migration amplitude to at least one radio transmitter. A common packaging could contain the tension sensor and the radio transmitter. In one example, the tension sensor is located at a node connecting the anchor residing in a body cavity with the intestinal sleeve. In another example, the tension sensor is located on a rigid lever connecting the anchor residing in a body cavity with the gastrointestinal sleeve.

In still a further example, at least one tension sensor is located between the pylorus anchor's windings.

Presented is also a method supporting correction of the intestinal sleeve and pylorus anchor location.

### LIST OF FIGURES AND THEIR BRIEF DESCRIPTION

FIG. 1 is an example of an existing implantable device;

FIG. 2 is an example of the present implantable device;

FIG. 3 is another example of the present implantable device;

FIG. 4 is a further example of the present implantable device; and

FIG. 4A is a detail illustrating a location of a tension sensor between the windings of the pylorus anchor.

### DESCRIPTION

Gastrointestinal operations combine the insertion of a restrictive gastric element anchored in a stomach and an extended tubular intestinal sleeve residing in the intestines. The food is routed into the intestines and passes through the tubular intestinal sleeve. The sleeve lining the internal walls of the intestines causes a particular effect of nutrients malabsorption. One of the difficulties encountered in placing the sleeve inside the gastrointestinal tract's lumen is that the intestinal sleeves tend to migrate. The intestinal sleeve does not adhere to the walls of the intestines. The pylorus anchor fixing the intestinal sleeve weakens in time, and the sleeve, together with the pylorus anchor, migrates into the intestine. The intestinal sleeve migration could cause small intestine blockage and apply excessive pressure to the intestine walls. The excessive pressure could cause the anchors associated with the sleeve to perforate the intestine walls.

The stomach contractions and changes in the patient's position (e.g., the patient is walking, sitting down, lying down, becoming involved in sporting activities, etc.), cause the sleeve and the pylorus anchor to rotate and exert additional pressure on the pylorus. The additional pressure on the pylorus increases the food absorption rate and complicates the device extraction. Removal of the intestinal sleeve through laparoscopic or open surgery means depends on the migrated sleeve and pylorus location and the damage that the migration caused to the gastrointestinal (GI) tract.

An intestinal sleeve that, in the course of its use, does not migrate and maintains the original position or at least supports monitoring of the intestinal sleeve location would significantly improve the use of such sleeves and simplify the intestinal sleeve post insertion treatment.

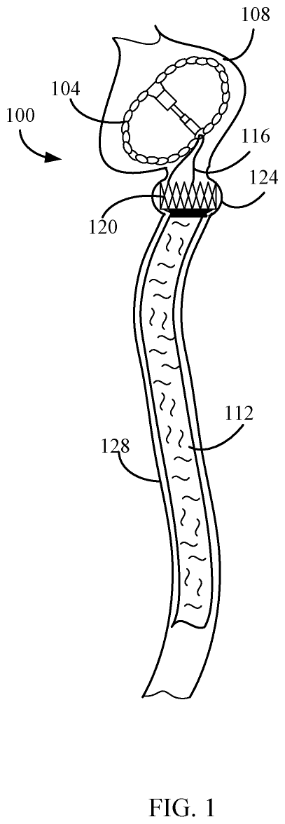

FIG. 1 illustrates currently commercially available obesity and diabetes treatment implantable device such as the one disclosed in the U.S. Pat. Nos. 10,368,976, 10,631,971, 10,327,938, and Pending Application for Patent No. 20200100888 all to the same assignee. Device (100) includes an anchor (104) residing in a stomach (108) and intestinal sleeve (112) attached with one or more sleeve cords (116) to anchor (104). Anchor (104) is illustrated as a bracelet-type anchor, although other known types of anchors could be used. The intestinal sleeve (112) in one of its ends, typically the end proximal to the stomach (108), includes a pylorus anchor (120), which positions and fixes the intestinal sleeve (112) in place. In some examples, the pylorus anchor (120) could reside in the stomach (108). In other examples, the pylorus anchor (120) location is proximal to a pylorus (124) or supported by pylorus (124). In addition to the intestine sleeve (112), pylorus anchor (120) support, sleeve cords (116) link the intestine sleeve (112) residing in the intestine (128) to anchor (104) residing in the stomach.

The obesity and diabetes treatment device (100) is inserted in a body cavity intraorally in a flexible or folded configuration. Anchor (104) and pylorus anchor (120), following the insertion, are converted from the flexible configuration into a rigid configuration. In their rigid configuration, anchor (104) and pylorus anchor (120) constrain the implantable devices' movement in a body cavity. Pyloric anchor (120) fixes the intestinal sleeve (112) at the entrance to the intestines. Tetrafluoroethylene or Polytetrafluoroethylene (PTFE), known under the trade name Teflon® are the materials from which walls of the intestine sleeve (112) are produced. Teflon® is a material non-permeable by the chime and other products of stomach activity. The walls of the intestine sleeve (112) could have a thickness of 20 to 50 microns. Intestinal sleeve (112) could be made of the desired length L, for example, 50 cm, 80 cm, or shorter or longer.

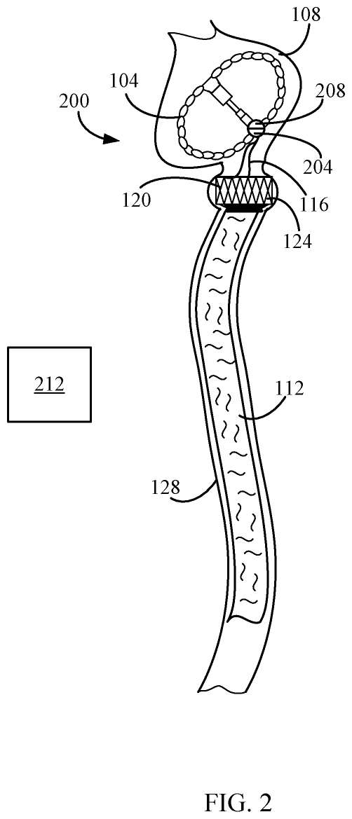

FIG. 2 is an example of the present implantable device. Implantable device (200) includes the components of the implantable device (100). Additionally, in one example, an intestinal sleeve cord (or cords) (116) attaches the intestinal sleeve (112) to anchor (104) through a tension sensor (204). Tension sensor (204) is located at a connecting node of anchor (104) residing in a body cavity with the pylorus anchor (120) of the intestinal sleeve (112). Tension sensor (204) senses tension caused by migration of the intestinal sleeve and the pylorus anchor (120) when such migration occurs. A suitable tension sensor could be such a sensor as a tension sensor (204) that includes an RFID (Radio Frequency Identification Device) element (208) communicating with the tension sensor (204). The RFID transmitter or element (208) could be a passive element that communicates with an RF receiver (212) located outside a patient's body. Tension sensor (204) and RFID (Radio Frequency Identification) element (208) could be packaged in the same package.

Tension sensor (204) measures the tension or force applied by a sliding intestine sleeve (112) and/or by pylorus anchor (120) to the tension sensor (204). The force caused by changes in the location of pylorus anchor (120) and intestine sleeve (112) affects the strain applied to sleeve cord/s (116) and accordingly to tension sensor (204). As the intestinal sleeve (112) and pylorus anchor (120) migrate into the intestine (128), the strain in the sleeve cords (116) grows. The value of the strain or force sensed by tension sensor (204) could indicate the migration amplitude or value of intestinal sleeve (112) migration. In some examples, a calibrating device based on the measurements of the strain could assess the amplitude of the migration could be included in the RFID transmitter or tension sensor.

A suitable tension sensor could be a FlexiForce Sensor or Single point of force sensor, both available commercially from Tekscan, Inc., South Boston MA02127 U.S.A. Alternatively, a QLA 414 Nano-in-line tension sensor commercially available from Futek, Inc., Irvine, CA 92618, U.S.A. could be used.

The RFID transmitter or element (208) of the tension sensor (204) could be in continuous communication with an RF receiver (212) or transmit the information in preset activation or transmission periods. The RFID element (208) of the tension sensor (204) could also become active and communicate with RF receiver (212) in an alarm mode when the strain or tension exceeds a preset magnitude of strain.

Based on the received strain value, the RFID element (208) of implantable device (200) could initiate communication with the user or caregiver to decide on the correction actions. User or caregiver alerting could be performed when the strain exceeds a preset value by a visual or audible signal. Intestinal sleeve and anchor migration is a long-term process. Following an earlier warning, the process can be stopped. The caregiver can interfere in the intestinal sleeve (112) migration process in the earlier stages when removing the intestinal sleeve or sleeve reposition is more comfortable to perform.

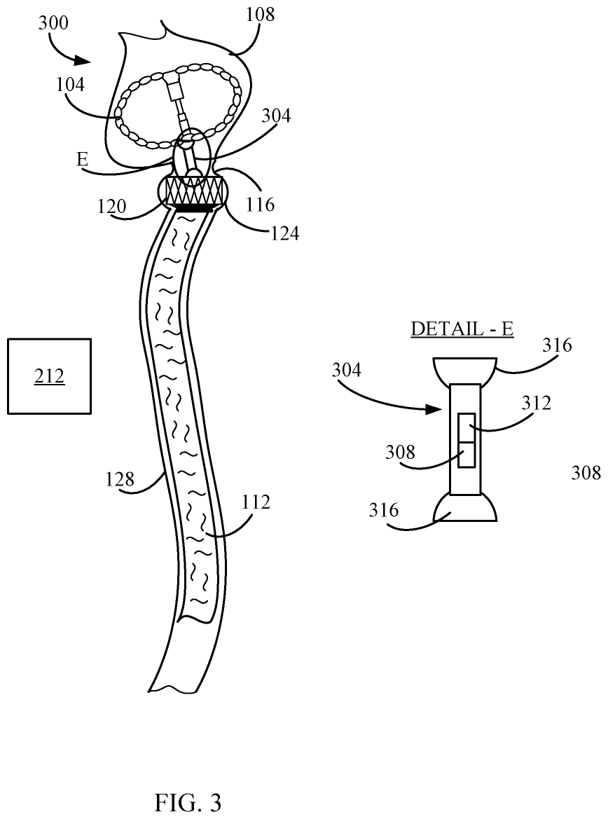

FIG. 3 is another example of the present implantable device. In the implantable device (300), a rigid lever (304) replaces the flexible sleeve cord (116) (FIG. 1). A rigid lever (304) includes a built-in tension sensor (308) and an RFID transmitter or element (312) communicating with the tension sensor (308). Rigid lever (304) mechanically connects with anchor (104) and pylorus anchor (120) with the help of spherical or ball joints (316). The ball joints support rotation of the implantable device (300) components on almost any angle relative to other device (300) components. Ball joints (316) limit the axial movement of the components. Migration of pylorus anchor (120) and intestine sleeve (112) in the axial direction (towards the intestine (128)) induce strain in the built-in tension sensor (308) that communicates the strain to the RFID element or transmitter (312) and further to the external RF receiver (212).

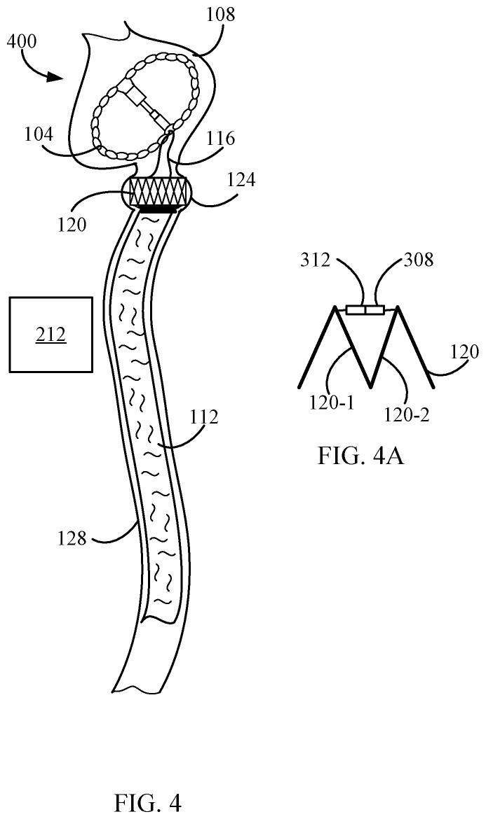

FIG. 4 is a further example of the present implantable device. Migration of the intestinal sleeve (112) with pylorus anchor (120), typically in the direction of the intestines, is characterized by a change in the shape and dimensions of pylorus anchor (120) that has to pass pylorus (124). A tension sensor (308) with an RFID transmitter or element (312) could be incorporated between two or more neighbor pylorus anchor (120) windings. Changes in pylorus anchor (120) dimensions induce strain in the pylorus anchor windings or between the pylorus anchor's (120) windings (120-1) and (120-2). Tension sensor (408) located between the pylorus anchor windings is configured to measure the strain caused by changes in the pylorus anchor (120) dimensions and communicate the strain values or amplitude to the RFID transmitter or element (412). RF element (412) communicates the status of the intestinal sleeve 112 to an external receiver (212) that could initiate communication with the user or caregiver that have to decide on the correction actions.

Several examples have been described. Nevertheless, it will be understood that various modifications may be made without departing from the implantable device's spirit and scope, and method of use.

Accordingly, other examples are within the scope of the following claims.

1-14. (canceled)

15. A gastrointestinal device, comprising:
an anchor residing in a body cavity;
an intestinal sleeve configured to be conducted down through intestines, the intestinal sleeve including, at a proximal end thereof, a pylorus anchor;
a cord connecting the anchor residing in the body cavity with the intestinal sleeve;
at least one tension sensor; and
at least one radio wave transmitter;
wherein the at least the one tension sensor senses tension caused by migration of the intestinal sleeve and the pylorus anchor, and communicates a migration value to at least one radio transmitter.

16. The gastrointestinal device of claim 15, wherein the body cavity is a stomach.

17. The gastrointestinal device of claim 15, wherein the cord connecting the anchor residing in the body cavity with the intestinal sleeve, is one of a group of cords consisting of a flexible sleeve cord and a rigid lever.

18. The gastrointestinal device of claim 17, wherein the at least one tension sensor is located in the rigid lever.

19. The gastrointestinal device of claim 15, wherein the at least one tension sensor is located at a node connecting the anchor residing in the body cavity with the intestinal sleeve.

20. The gastrointestinal device of claim 15, wherein the pyloric anchor comprises a plurality of windings and the at least one tension sensor is located between the windings.

21. The gastrointestinal device of claim 15, wherein the migration value comprises a migration amplitude, and wherein the at least one tension sensor is configured to communicate the migration amplitude to at least one calibrating device.

22. The gastrointestinal device of claim 21, wherein the at least one tension sensor is configured to continuously sense and monitor a location of the intestinal sleeve.

23. An intestinal sleeve, comprising:
a flexible tubular sleeve open on both ends;
a pylorus anchor incorporated in one of the ends of the flexible sleeve, the pylorus anchor comprising a plurality of windings and a tension sensor located between the windings, the tension sensor configured to sense and communicate a force acting on the pylorus anchor.

24. The intestinal sleeve of claim 23, wherein the tension sensor is configured to sense strain induced on the pylorus' anchor's windings due to changes in dimensions of the pylorus anchor.

25. A method of treating obesity by a gastrointestinal device, comprising:
inserting intraorally into intestines a gastrointestinal device including at least one tension sensor and one RFID transmitter;
fixing an intestinal sleeve at the entrance to the intestines;
employing at least one tension sensor and continuously monitoring a strain applied by the intestinal sleeve and pylorus anchor caused by migration of the intestinal sleeve and pylorus anchor; and
when a value of the strain caused by migration of the intestinal sleeve and pylorus anchor exceeds a preset strain value, providing a warning to a caregiver.
